(19) United States
(12) Patent Application Publication
(10) Pub. No.: US 2009/0042869 A1
(43) Pub. Date: Feb. 12, 2009

(54) SIGNAL-DEPENDENT SPLICING OF TISSUE FACTOR PRE-mRNA IN PLATELET CELLS

(75) Inventors: Andrew S. Weyrich, Salt Lake City, UT (US); Hansjorg Schwertz, Salt Lake City, UT (US); Guy Zimmerman, Salt Lake City, UT (US); Neal Tolley, Salt Lake City, UT (US)

Correspondence Address: MORRISS OBRYANT COMPAGNI, P.C., 734 EAST 200 SOUTH, SALT LAKE CITY, UT, 84102, US

(73) Assignee: UNIVERSITY OF UTAH RESEARCH FOUNDATION, Salt Lake City, UT (US)

(21) Appl. No.: 12/143,645

(22) Filed: Jun. 20, 2008

Related U.S. Application Data
(60) Provisional application No. 60/936,593, filed on Jun. 20, 2007.
(60) Provisional application No. 60/936,528, filed on Jun. 20, 2007.

(51) Int. Cl. A61K 31/54; C12Q 1/68; C40B 30/06; G01N 33/00; A61P 7/00; C40B 30/08; C12Q 1/02

(52) U.S. Cl. 514/224.2; 435/6; 506/10; 436/86; 506/11; 435/29

(57) ABSTRACT

The invention relates to therapeutic target recognition, development, and validation of a compound capable of directly or indirectly modulating TF pre-mRNA splicing in a platelet cell and the use of a platelet TF pre-mRNA splicing modulator for the treatment of a subject suffering from, or thought to be suffering from, disordered coagulation.

### CROSS-REFERENCE TO RELATED APPLICATIONS

This application claims the benefit of U.S. Provisional Patent Application No. 60/936,593, filed Jun. 20, 2007, which is related to U.S. Provisional Application No. 60/936,528, filed Jun. 20, 2007, the entirety of each of which is incorporated by reference.

### STATEMENT REGARDING FEDERALLY SPONSORED RESEARCH OR DEVELOPMENT

This invention was made with Government support under HL-66277, HL-44525, HL-75507, and/or HL-48872, awarded by the National Institutes of Health. The U.S. Government has certain rights to this invention.

### TECHNICAL FIELD

The invention relates to biotechnology generally and more particularly to the use of Tissue Factor (TF) pre-mRNA splicing, TF-dependent coagulation and/or stabilization of a platelet thrombus to identify a therapeutic agent and the use of a therapeutic agent for the treatment of a subject suffering from, or thought to be suffering from, disordered coagulation.

### BACKGROUND

The following discussion of the background of the invention is provided to aid the reader in understanding the invention and is not an admission that anything herein describes or constitutes prior art.

Platelets are anucleate cells that are derived from bone marrow megakaryocytes and circulate in the blood. They are best known for their ability to bind to exposed subendothelial matrix at sites of vascular injury and aggregate with one another to form a primary hemostatic plug^{1,2} that prevents or limits bleeding. Platelet hemostatic responses involve activation by the coagulation protein thrombin, which is recognized by a cell surface receptor, or by other agonists. This results in a rapid and complex series of events that include changes in the shape of the platelets, inside-out signaling of integrins, the release of preformed or internalized proteins from subcellular granules, and generation of eicosanoids by constitutive enzymes.

Phosphatidyl serine (PS) exposure on the surface of activated platelets provides a thrombogenic surface for the activation of the coagulation protease cascade^{3,4}. PS-exposed membranes enhance the catalytic efficiency of membrane-anchored tissue factor (TF)⁵, the primary initiator of the coagulation cascade that leads to the conversion of prothrombin to thrombin and fibrinogen to fibrin⁶. Whether circulating platelets intrinsically possess TF protein, however, is a debated question⁷⁻¹². Studies have demonstrated that monocytederived microparticles bind to activated platelets¹³⁻¹⁵ and activated platelets themselves release TF-positive microparticles^{9,16,17}.

Tissue Factor, a 47-kDa glycoprotein, is a transmembrane receptor for plasma coagulation factor VIIa (fVIIa) and formation of TF/fVIIa complexes on the cell surface triggers the coagulation cascade at least in part by activating coagulation factors IX and X. The resultant protease factor Xa then cleaves prothrombin to produce thrombin, which in turn converts fibrinogen into a fibrin matrix.

Although platelet activation to prevent bleeding is of paramount importance, platelets have other roles as well. Platelet adhesion and aggregation in the flowing blood are critical in atherosclerosis and syndromes of pathologic thrombosis. They also influence vascular growth, permeability, and integrity, regulate metastasis of certain tumor types, and are intimately involved in inflammation.

Disordered coagulation is a frequent hematological complication, with thromboembolic disease affecting approximately 15% of all cancer patients. Disordered coagulation also includes superficial and deep venous thrombosis, pulmonary emboli, thrombosis of venous access devices, arterial thrombosis and embolism.

A number of platelet aggregation inhibitors or anticoagulants have been developed or are currently being developed, including, but not limited to, the glycoprotein (GP) IIb/IIIa receptor antagonists and clopidogrel (Plavix), which now play a pivotal role in cardiology and clinical medicine. However, these inhibitors or anticoagulants also pose a serious challenge for all health care personnel involved in their management. This challenge largely stems from the need to prevent thrombosis on the one hand and the need for therapeutic hemostasis at surgical sites or other sites of vascular injury on the other.

Platelet aggregation inhibitors or anticoagulants include antagonists of the GP IIb/IIIa receptor, such as the monoclonal antibody abciximab (formerly called c7E3 Fab) (approved for use in percutaneous coronary intervention (PCI)), Tirofiban (a nonpeptide approved for treatment of acute coronary syndromes, such as unstable angina and myocardial infarction) and eptifibatide (a peptide approved for use in both PCI and acute coronary syndromes) (see, EP418316, U.S. Pat. No. 5,292,756, and EP477295, respectively).

Platelet aggregation inhibitors or anticoagulants also include orally administered thienopyridine antiplatelet agents, such as Ticlopidine and clopidogrel (see, U.S. Pat. Nos. 4,015,141, and 4,847,265), which may be useful for stroke or recent transient ischemic attack, subacute stent thrombosis, unstable angina, peripheral arterial disease, saphenous vein coronary bypass grafts, myocardial infarction, stroke, peripheral arterial disease and diabetic retinopathy. However, ticlopidine (TICLID™) and clopidogrel (PLAVIX™), are associated with potentially fatal thrombotic thrombocytopenic purpura (TTP).

Despite the development of platelet aggregation inhibitors or anticoagulant agents, aspirin, an irreversible inhibitor of platelet cyclooxygenase activity, remains the standard against which other drugs are judged. Yet, even asprin has undesirable side effects, including intolerance, allergy, resistance, peptic ulceration, and intracranial hemorrhage.

Therefore, there exists a substantial need for an improved therapeutic agent useful in preventing or modulating coagulation in a subject.

### SUMMARY OF THE INVENTION

The invention relates to a method of identifying a compound capable of inhibiting pre-mRNA splicing or RNA splicing dependent coagulation activity in a platelet cell, comprising contacting a platelet cell with a compound and measuring coagulation and/or pre-mRNA splicing in the platelet cell. In an exemplary embodiment, the invention relates to tissue factor (TF) pre-mRNA splicing or TF-dependent coagulation activity in a platelet cell and in another exemplary embodiment the invention relates to IL-1β pre-mRNA splicing or IL-1β-dependent coagulation activity.

The invention also relates to the use of a platelet pre-mRNA splicing modulator for the treatment of a subject suffering from, or thought to be suffering from, disordered coagulation, comprising administering an effective amount of the platelet pre-mRNA splicing modulator to the subject. In an exemplary embodiment, the invention relates to the use of Tg003 for the purpose of reducing TF pre-mRNA splicing in platelet cells of a subject and treating disordered coagulation. In another exemplary embodiment, the pre-mRNA splicing modulator is a TF pre-mRNA splicing modulator and in another exemplary embodiment the pre-mRNA splicing modulator is an IL-1β pre-mRNA splicing modulator.

In an exemplary embodiment, the invention relates to a method of screening a compound for modulation of Clk1 activity. In another exemplary embodiment, the invention relates to a method of screening a compound for modulation of pre-mRNA splicing and the production of mature mRNA. In another exemplary embodiment, the invention relates to a method of screening a compound for modulation of pre-mRNA-dependent coagulation of platelet cells. Exemplary pre-mRNAs that may be utilized in a screening method include TF pre-mRNA and IL-1β pre-mRNA.

The invention also relates to methods of regulating thrombus formation associated with surgery, microsurgery, angioplasty, or trauma or to inhibit thrombus formation and other functions of TF in abnormal haemostatic conditions associated with a disease (such as deep vein thrombosis, disseminated intravascular coagulation (DIC), coronary artery disease, sepsis, inflammation, atherosclerosis, and/or cancer). In an exemplary embodiment, the invention relates to a method of treating disregulated coagulation in a subject by administering a compound that modulates Clk1 and/or TF pre-mRNA splicing, thereby modulating thrombosis in the subject.

The invention also relates to the use of a compound capable of inhibiting TV pre-mRNA splicing or TF-dependent coagulation activity in a platelet cell for the manufacture of a medicament for the treatment of disordered coagulation. The invention also relates to the use of Tg003 ((Z)-1-(3-Ethyl-5-methoxy-2,3-dihydrobenzothiazol-2-ylidene)propan-2-one) for the manufacture of a medicament for the treatment of disordered coagulation.

### BRIEF DESCRIPTION OF THE DRAWINGS

FIG. 1 illustrates the absence of contaminating Leukocytes in the platelet preparations. Flow cytometric analysis for CD45 (bottom) or control IgG (top) in leukocyte-depleted platelets (Control Platelets) or monocytes. For this analysis, antigen expression was screened on all of the cells in the preparation (not depicted) and corroborates previous results demonstrating that CD45-depleted washed platelet preparations are devoid of contaminating leukocytes¹⁸.

FIG. 2A shows the presence of mature TF mRNA in activated, but not freshly isolated, platelet cells.

FIGS. 2B and 2C show a time course for the processing of TF pre-mRNA to mature TF RNA in activated platelet cells.

FIG. 2D shows the results of indirect in situ PCR for intronic TF pre-mRNA in megakaryocytes (left) and megakaryocytes with proplatelet extensions (right). In the bottom panels (no RT), the reverse transcriptase (RT) was omitted from the RT reaction.

FIG. 2E shows the results of direct in situ PCR for TF pre-mRNA in quiescent platelets (top left), whereas PCR for mature TF mRNA was conducted in platelets adherent to fibrinogen in the presence of thrombin for 1 h (top right). In the bottom panels (no RT), the reverse transcriptase was omitted during the RT reaction.

FIG. 3 shows that activated platelets express full-length, mature human tissue factor mRNA (mHTF). Platelets were left quiescent (0) or adhered to fibrinogen in the presence of thrombin for 4 h (240). Long-range PCR, using primers that targeted sequences in exon 1 and exon 6, were used to analyze the expression pattern of mHTF in mRNA isolated from activated platelets.

FIGS. 4A and 4B show that quiescent platelets exposed to different agonists splice tissue factor (TF) pre-mRNA into mature message. FIG. 4A shows TF and GAPDH mRNA expression in freshly isolated platelets (control), platelets that are activated in suspension by ADP (20 μM), or platelets adherent to fibrinogen in the presence of thrombin for the designated times. FIG. 4B shows TF mRNA expression in platelets that were left in suspension (control), platelets adherent to fibrinogen in the presence of thrombin, or platelets that are activated in suspension by ADP (20 μM), collagen (10 μg/ml), or thrombin (0.05 U/ml) for 30 min.

FIGS. 5A and 5B illustrate the increase in TF-dependent procoagulation activity during platelet activation. FIG. 5A illustrates a timecourse from 0 to 60 minutes of TF-dependent procoagulant activity in platelets that have adhered to fibrinogen in the presence of thrombin. In FIG. 5B, each line represents procoagulant activity from platelet membranes (Pits), platelet-derived microparticles (Mp), or platelet membranes together with microparticles from the same sample (Plts+Mp) over a time course from 0 to 4 hours. The data in FIGS. 5A and 5B are displayed as pM of TF per 2×10⁹ platelets and represent the mean±SEM of three independent experiments.

FIG. 5C shows immunolocalization of TF in freshly isolated platelets (left) and platelets that have adhered and spread on immobilized fibrinogen in the presence of thrombin for 2 h (right). The green stain in both panels represents actin. The red stain in the right panel shows immunolocalization of TF on the surface of activated platelets (see arrow).

FIG. 5D illustrates, bars (n 4), TF-dependent procoagulant activity in freshly isolated platelets or platelets activated as in FIG. 5B for 4 h in the presence or absence of factor VIIa. A single asterisk (*) indicates a statistically significant difference (P<0.05) in TF-dependent procoagulant activity between freshly isolated (baseline) and activated platelets; the double asterisk (**) represents a significant difference (P<0.05) between activated platelets under untreated or treated conditions.

FIG. 5E illustrates, bars (n 4), TF-dependent procoagulant activity in freshly isolated platelets or platelets activated as in FIG. 5B for 4 h in the presence or absence of a neutralizing antibody directed against TF. Statistical significance is as described in FIG. 5D.

FIG. 5F illustrates clot formation in plasma that is incubated with membranes isolated from quiescent platelets or platelets activated with fibrinogen and thrombin for 5 min (left) or 2 h (right). The white bars represent activated platelets that were incubated with a neutralizing antibody directed against tissue factor (Anti-TF). The data in FIGS. 5D, 5E, an 5F represent the mean±SEM of six independent experiments.

FIG. 6A shows immunolocalization of Megakaryocytes with proplatelet extensions stained with anti-Clk1 (top) or control IgG (bottom). The right panels are overlays where anti-Clk1 or IgG are in red, wheat germ agglutinin is in green, and topro-3, which stains nuclei, is in blue.

FIG. 6B shows immunolocalization of Clk1 in quiescent platelets (top) or platelets activated by adherence to immobilized fibrinogen in the presence of thrombin for 1 h (middle and bottom). The cells were stained with anti-Clk1 (top and middle) or control IgG (bottom). The right panels are overlays in which anti-Clk1 or IgG is in red, polymerized actin is in green, and colocalization of the two markers is identified by yellow staining.

FIG. 6C illustrates a time course of Clk1-dependent SF2/ASF phosphorylation in activated platelet lysates.

FIG. 6D illustrates Clk1-dependent SF2/ASF phosphorylation in freshly isolated platelet lysates or platelets that were pretreated with vehicle or Tg003 (Clk Inh) (lanes 2-5). The bars for FIG. 6C and FIG. 6D represent fold increases in SF2/ASF phosphorylation over baseline as estimated by densitometry. Platelets in FIGS. 6C and 6D were activated as in 6B, lysed, and immune complex kinase assays for Clk1 activity were performed with recombinant SF2/ASF as the substrate.

FIGS. 7A and 7B show that SF2/ASF phosphorylation is specifically mediated by Clk1-dependent mechanisms. In FIG. 7A platelets were adhered to immobilized fibrinogen in the presence of thrombin for 30 min, the cells were then lysed, and the proteins were captured with control IgG or an antibody against Clk1. Immune-captured complexes were subsequently used to induce SF2/ASF phosphorylation. FIG. 7B shows Clk1-dependent SF2/ASP phosphorylation in quiescent platelet lysates (0) or platelets that were pretreated with or without Tg003 (Clk Inh) before activation (lanes 2 and 3). The last lane is a Clk-immunoprecipitated complex that was assayed in the absence of recombinant SF2/ASF. The bars for this figure represent fold increases in SF2/ASF phosphorylation over baseline as estimated by densitometry.

FIGS. 8A-D show that interruption of Clk1 blocks signal-dependent TF pre-mRNA splicing and bioactive protein accumulation in platelets. FIG. 8A shows human TF pre-mRNA (pHTF; 904 bp) and mature TF mRNA (mHTF; 297 bp) in freshly isolated platelets and in platelets adherent to immobilized fibrinogen and coactivated with thrombin for 2 h. The activated platelets were either left untreated (lane 3) or pretreated with vehicle (Veh) or Tg003 (Clk Inh). FIG. 8B is a western blot analysis showing TF protein expression in platelets and platelet-derived microparticles. The platelets were left quiescent (baseline) or adhered to immobilized fibrinogen and coactivated with thrombin for 30 min or 4 h in the presence or absence of Tg003 (Clk Inh). Recombinant TF was used as a positive control. FIG. 8C depicts TF-dependent procoagulant activity in freshly isolated platelets or platelets activated, as in FIG. 2B, in the presence or absence of the Clk inhibitor. The data are displayed as pM of TF per 2×10⁹ platelets and represent the mean±SEM of six independent experiments. A single asterisk (*) indicates a statistically significant difference (P<0.05) in TF-dependent procoagulant activity between freshly isolated (baseline) and activated platelets; the double asterisk (**) represents a significant difference (P<0.05) between activated platelets under untreated or treated conditions. FIG. 8D shows plasma clot formation in the presence of membranes isolated from freshly isolated platelets (Control) or platelets activated for 5 min (left) or 2 h (right) with fibrinogen and thrombin. The activated platelets were pretreated with either the Clk inhibitor (Clk Inh) or DMSO. The data represent the mean±SEM of five independent experiments. The single asterisk (*) indicates a statistically significant difference (P<0.05) in the rate of clot formation in plasma samples exposed to fibrinogen and thrombin-treated platelets compared with quiescent platelets (Control) and activated platelets treated with the Clk inhibitor.

FIGS. 9A and 9B show that inhibition of translation prevents activated platelets from generating bioactive TF. FIG. 9A illustrates a representative timecourse (i.e., 0-60 min) of TF-dependent procoagulant activity in untreated or puromycin-treated platelets that have adhered to fibrinogen in the presence of thrombin. The data are displayed as pM of TF per 2×10⁹ platelets. FIG. 9B shows TF-dependent procoagulant activity in freshly isolated platelets or platelets adherent to immobilized fibrinogen and coactivated with thrombin for 60 min. in the presence or absence of puromycin. The bars indicate the mean±SEM of four independent experiments. A single asterisk (*) indicates a statistically significant difference (P<0.05) in TF-dependent procoagulant activity generated by activated platelets compared with freshly isolated platelets (baseline). The double asterisk (**) indicates a statistically significant difference (P<0.05) between activated platelets treated with or without puromycin.

FIG. 10 shows that inhibition of Clk1 does not adversely affect other platelet functional responses. Platelets were adhered to immobilized fibrinogen and coactivated with thrombin in the presence or absence of Tg003 (Clk Inh). After 1 h, the cells were prepared for immunocytochemical detection (red staining) of Clk1 (top) or β-tubulin (bottom). The green stain in each panel identifies polymerized actin. This figure is representative of three independent experiments and demonstrates that Tg003 does not block adherence, spreading, actin polymerization, or β-tubulin organization in platelets that are exposed to immobilized fibrinogen in the presence of thrombin.

FIG. 12 illustrates a proposed model by which platelet-derived tissue factor contributes to propagation and stabilization of a thrombus.

### DETAILED DESCRIPTION OF THE INVENTION

As used herein, “sample” means any sample of biological material derived from a subject such as, but not limited to, blood, plasma, mucus, and biopsy specimens and fluid which has been removed from the body of the subject. The sample which is tested according to the method of the invention may be tested directly or may require some form of treatment prior to testing. For example, a blood sample may require isolation of platelet cells prior to testing. Further, to the extent that the biological sample is not in liquid form, (for example it may be a solid, semi-solid or a dehydrated liquid sample) it may require the addition of a reagent, such as a buffer, to mobilize the sample.

As used herein, “blood” means whole blood or any fraction thereof for example plasma, platelets, or a concentrated suspension of cells.

As used herein, “disordered coagulation” includes, but is not limited to, thromboembolic disease, intravascular thrombosis, microvascular platelet thrombosis, venous thromboembolism, deep vein thrombosis, disseminated intravascular coagulation (DIC), coronary artery disease, fibrinolysis, and/or sepsis.

As used herein, “modulation” or “modulator” means inducing an increase or decrease in the amount of TF-dependent coagulation, TF pre-mRNA splicing or Clk1 activity, particularly in response to one or more signals.

As used herein, “subject” means a mammal, including, but not limited to, a human, horse, bovine, dog, or cat.

As used herein, “therapeutic target recognition, development, and validation” means any method which enables an artisan to recognize, develop, or validate the efficacy of a therapeutic agent which displays an interaction with Clk1 and/or affects TF mRNA splicing.

As used herein, “TF pre-mRNA splicing” means signal dependent removal of at least one intronic sequence from a pre-existing RNA within a platelet cell, and preferably removal of all intronic sequences so as to produce a mature mRNA capbable of being translated into TF protein. TF pre-mRNA splicing may be measured directly, for example, by PCR, or indirectly, for example, by measuring TF-dependent coagulation activity, TF protein production or Clk1 activation.

The invention relates to the finding that platelets from healthy human subjects contain TV pre-mRNA and process it to the mature transcript in response to cellular activation. As a result activated platelets produce TF protein, have procoagulant activity, and accelerate plasma clot formation. The intracellular signaling pathway that controls TF pre-mRNA splicing has been found to involve a Cdc2-like kinase, Clk1, an enzyme not previously known to be present or operate in platelets. Inhibition of Clk1 signaling in activated platelets blocks splicing factor 2 (SF2)/alternative splicing factor (ASF) phosphorylation, TF pre-mRNA splicing, and de novo accumulation of bioactive TF protein.

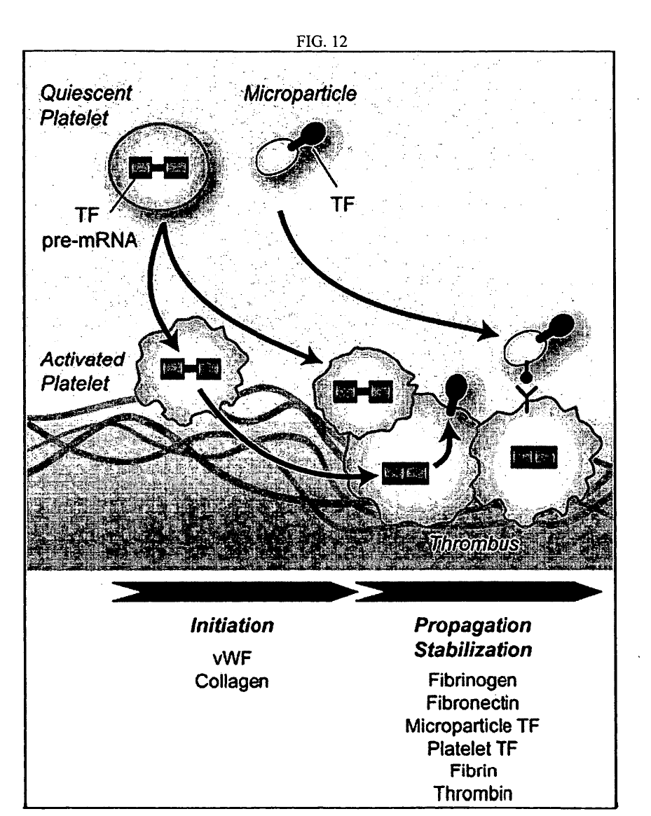

Pre-mRNA splicing and regulated translation of processed mRNAs are functions that allow activated platelets to alter their transcriptome and proteome in response to stimulation^{18,20,24}. The invention herein demonstrates that platelets also use their splicing machinery to control the expression of TF and identifies a new intermediate, Clk1, in the signaling pathway leading to TV synthesis. The results indicate that quiescent platelets contain TF pre-mRNA but do not express significant levels (P<0.05) of TV protein or activity under basal conditions. In contrast, activated platelets express both TF mRNA and bioactive TV protein. Pre-mRNA splicing and translation of TF message into protein is observed as early as 5 min after activation and is sustained for at least 4 h. The time scale of this response suggests that platelet-derived TF sustains the growth of the thrombus and increases its stability by enhancing fibrin deposition (see, FIG. 12). Formation of a stable thrombus is essential for hemostasis and promotes wound healing at the site of vascular injury^{25,26}. Tissue factor also modulates inflammation and angiogenesis^{27,28}, indicating that it affects prolonged functional responses.

Prior to the present invention, the relative contributions of platelet derived TF and TF-positive microparticles to thrombus formation under different pathologic conditions was not known. Furthermore, while on a per-cell basis lipopolysaccheride-stimulated monocytes generate greater amounts of IF than platelets, the number of circulating platelets far exceed (i.e., ˜500-1,000-fold greater) the number of monocytes per volume of blood. Thus, platelets have the potential to provide a far greater impact on thrombus formation than was previously known.

In addition, it has been found that pre-mRNA splicing is markedly increased in patients with sepsis, a clinical condition in which disordered coagulation is a central feature^{30,31}. TV mRNA expression patterns in platelets from septic patients were analyzed and within the first 24 hours of admission into the intensive care unit (ICU), platelets from over half of the patients expressed spliced TF mRNA and the incidence of splicing was increased in patients with high APACHE II scores, an index of severity of critical illness.

Furthermore, it was found that TF pre-mRNA splicing was more common in elderly patients than patients under the age of 65. This indicates that platelets from elderly subjects may have an enhanced tendency to splice TF pre-mRNA compared to platelets from young donors.

Therefore, TF pre-mRNA splicing alters the functions of platelets in clinical syndromes of inflammation and thrombosis. In addition, subjects receiving heparin and/or asprin exhibited TF pre-mRNA splicing. Hence, heparin and asprin do not appear to have a significant impact on TF pre-mRNA splicing.

The invention demonstrates that Clk1 modulates TF pre-mRNA splicing and that TF pre-mRNA splicing is a potential therapeutic target in syndromes of disordered coagulation. For example, the invention demonstrates that TF pre-mRNA splicing is increased during clinical conditions of disordered coagulation (e.g., sepsis) and in the elderly, which have increased risk of developing VTE. Therefore, the invention also provides a method of treating a subject having a condition or disease associated with disordered coagulation.

Furthermore, the invention provides a method of identifying or screening for Clk1 modulators or TF pre-mRNA splicing modulators, i.e., inhibitors (e.g., a Clk1 antagonist) or activators (e.g., a Clk1 agonist). While current platelet inhibitors constitute a broad spectrum of pharmaceutical agents that are being extensively used in medical applications, such as cardiovascular medicine, many of the commonly used platelet inhibitors do not modulate Clk1 activity and/or TF pre-mRNA splicing. As a result, the invention provides a method of therapeutic target recognition, development, and validation for compounds that modulate Clk1 and/or TF pre-mRNA splicing in platelet cells. More particularly, TF is known to trigger or play a substantial role in the coagulation cascade of several human diseases, such as sepsis and VTE, is present in atherosclerotic plaques, increases in coronary plaques and plays a role in coronary syndromes. Thus, the invention provides a method of therapeutic target recognition, development, and validation to identify therapeutic agents that may be useful in the treatment of disordered coagulation, such as sepsis, VTE, atherosclerotic plaques, coronary plaques and coronary syndromes.

In an exemplary embodiment, the invention relates to screening a compound for the ability to modulate Clk1 activity by measuring Clk1 activity²³ (e.g., phosphorylation rates), TF pre-mRNA splicing, or platelet aggregation. In another exemplary embodiment, the invention relates to the use of Tg003 to inhibit Clk1 activity. In yet another exemplary embodiment, the invention relates to the use of inhibitory oligonucleotides, e.g., RNA interference (iRNA), for the inhibition of TF pre-mRNA splicing and/or inhibition of platelet activation. For example, the use of inhibitory oligonucleotides to block splicing of the intron located between exons 4 and 5.

Platelet cells may be isolated by any method known in the art and used to screen compounds for the ability to modulate production of mature TF and/or Clk1 activity, which includes, Clk1 kinase assays, measuring pre-mRNA splicing and TF-dependent procoagulant activity. TF production may be measured by antibody binding and assayed by western blot, fluorescence activated cell sorting or other techniques known in the art.

The invention has particular relevance to conditions characterized by aberrant, unwanted, or otherwise inappropriate blood coagulation, which include, but are not limited to: haemostasis related disorders; hypercoagulate states, including inherited or acquired; thrombosis, including deep vein thrombosis; pulmonary embolism; thromboembolic complications associated with atrial fibrillation; cardiac valve replacement; coronary thrombolysis, for example, during acute myocardial infarction; percutaneous transluminal angioplasty; ischemia-reperfusion injury and post-operative thromboembolism.

According to the invention, at least one compound may be screened for the ability to modify the expression and/or function of Clk1, TF protein, TF-dependent coagulation activity, mature TF mRNA and/or TF pre-mRNA in or associated with platelet cells. This also makes it possible to screen a large number of compounds (a library of compounds) for the ability to modulate TF pre-mRNA splicing. A large number of screening methods are made available by the invention, for example, an antibody which is directed against Clk1 and/or TF may be employed in a detection method, such as ELISA (enzyme-linked-immuno sorbent assay), which is known to the skilled person. Other substances that may be used for the screening methods are oligonucleotides, which are suitable, for example, using the polymerase chain reaction (PCR), for detection of mature TF mRNA and/or TF pre-mRNA, either with or without amplification of the RNA or cDNA to be analyzed. Yet other substances that may be used for the screening methods are polypeptides, including antibodies, which are suitable for detection of Clk1 activity or activation, or production of TF protein (e.g., by ELISA or Western Blot). Alternatively, TF-dependent coagulation activity may be measured.

The invention also relates to a kit that provides the necessary components for a screening assay. This kit comprises at least one substance which is suitable for detecting the expression and/or function of Clk1, TF protein, TF-dependent coagulation activity, mature TF mRNA and/or TF pre-mRNA in platelet cells. The kit may also contain additional assay components (e.g., reagents), labels, and/or instructions.

Clk1 activity may be measured by any method known in the art. In an exemplary embodiment, Clk1 activity may be determined using an immune complex kinase assay. An antibody against Clk1 is used for immunoprecipitation of the protein. Non-immune rabbit IgG is used as a control, and in select experiments recombinant SF2/ASF is removed from the assays to screen for nonspecific incorporation of radiolabeled phosphate. Kinase assays are performed by addition of recombinant SF2/ASF (Protein One) in the presence of γ-[³²P]ATP (MP Biomedicals). At the end of this incubation period, the agarose beads and immune complexes are removed by centrifugation, and Clk1 activation is measured by SDS-PAGE.

For additional methodologies, see U.S. Pat. No. 7,045,289, and U.S. Patent Publications 20040197845, and 20060078559.

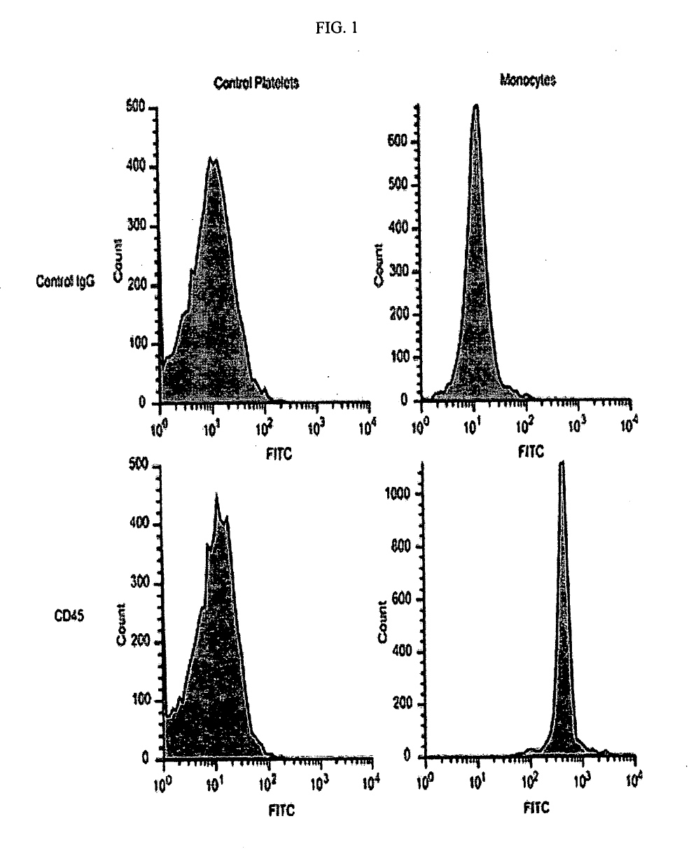

To address the question of whether or not platelets express TF, highly purified platelet preparations that were isolated from healthy volunteers¹⁸ were used to determined if platelets contained TF mRNA. The leukocyte-depleted preparations did not express CD45, PSGL-1, or CD14 (FIG. 1). For this analysis, antigen expression was screened on all of the cells in the preparation (not depicted) and corroborates previous results demonstrating that CD45-depleted washed platelet preparations are devoid of contaminating leukocytes. Similar results were observed when the cells were stained with conjugated antibodies directed against PSGL-1 or CD14 (not depicted).

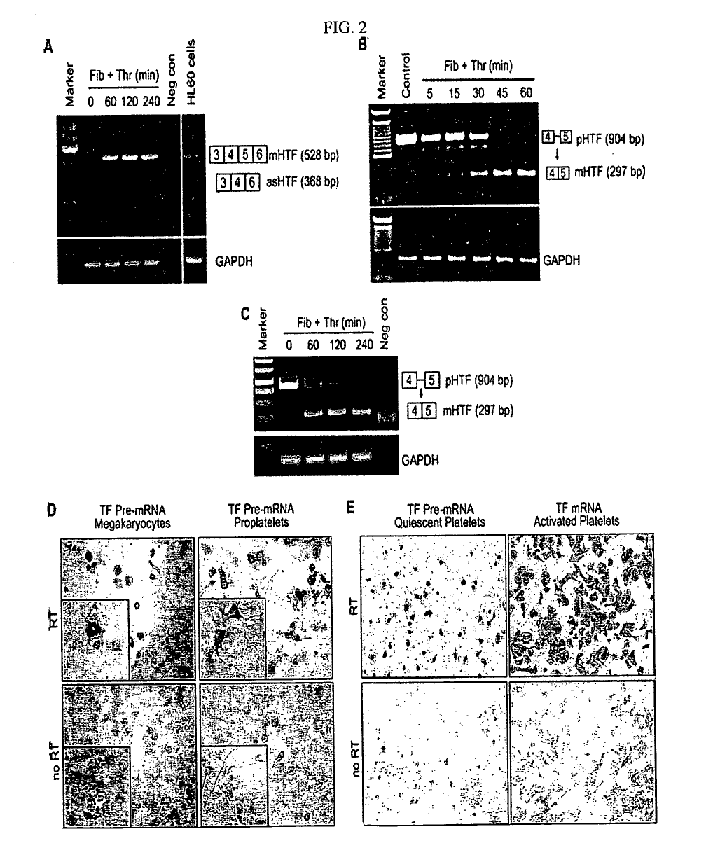

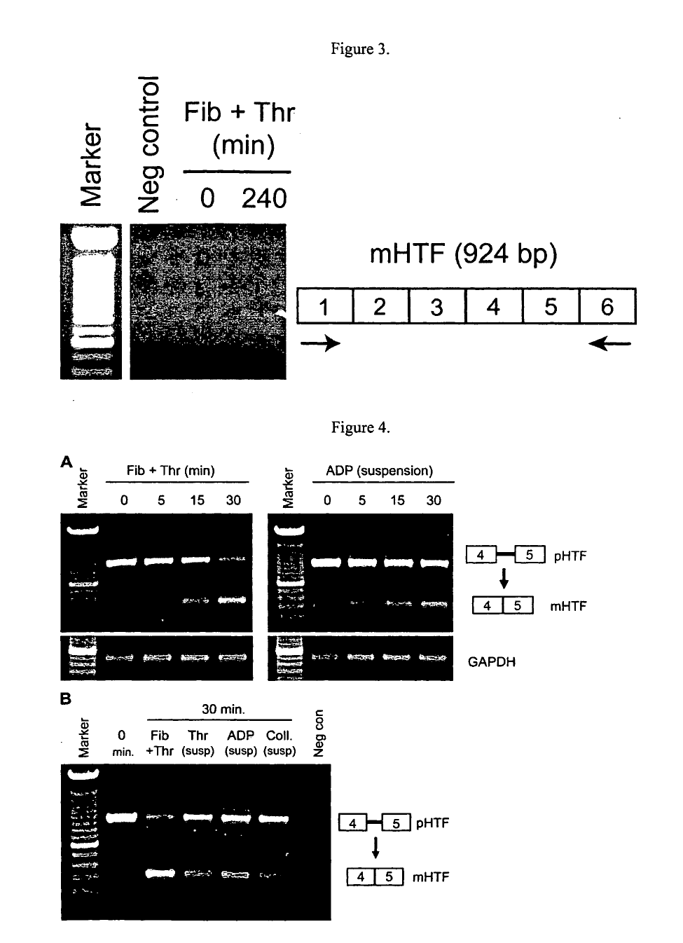

Unexpectedly, it was found that stimulated, but not quiescent, human platelets contain TF mRNA (FIG. 2A). Platelet cells were activated by adherence to fibrinogen and the addition of thrombin. The negative control lane (FIGS. 2A and 2C) is PCR without a cDNA template and GAPDH was used as an internal control. The alternatively spliced variant of TF¹⁹ was not detected in stimulated platelets under the conditions of this experiment; the variant was detected, however, in HL60 myeloid leukocytes or resting primary human monocytes (FIG. 2A). The fact that this message is mature human tissue factor was confirmed by amplification of the entire TF message, subsequent cloning and sequencing of the PCR product (FIG. 3). Platelets were either left quiescent (0) or adhered to fibrinogen in the presence of thrombin for 4 hours (240), and after isolation the mRNA was amplified using long-range PCR with primers targeting sequences in exon 1 and exon 6 (FIG. 3).

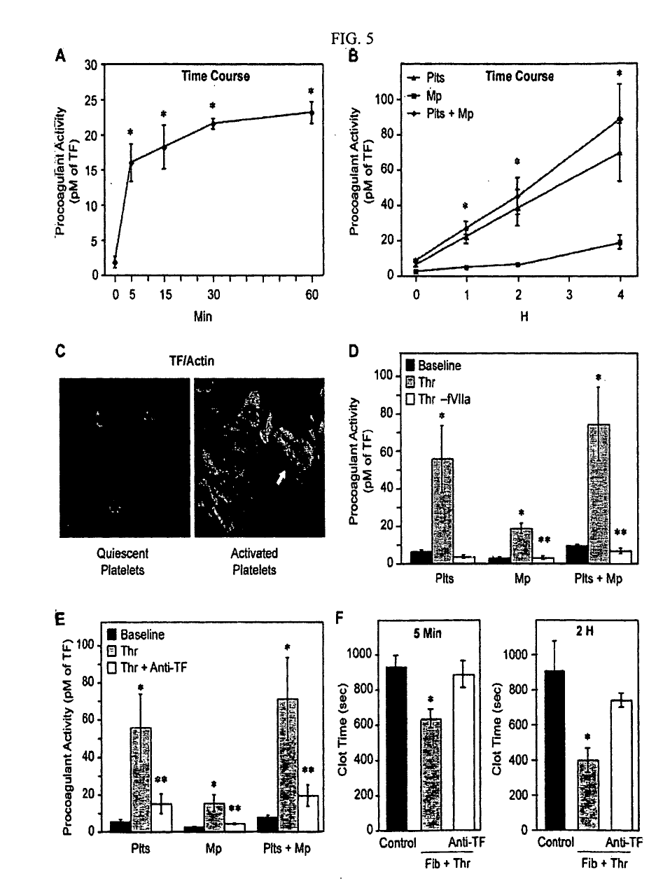

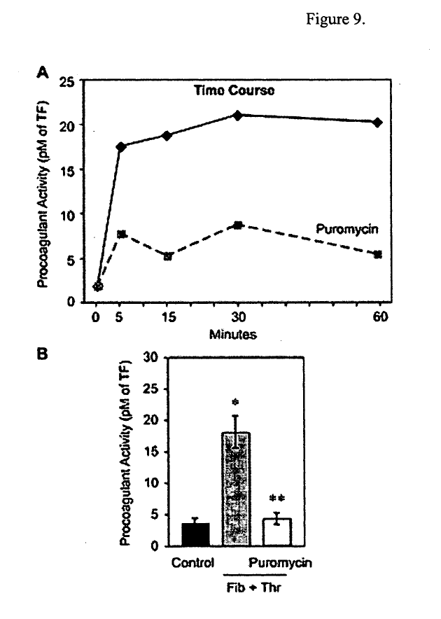

It was surmised that unstimulated platelets contain intron-rich TF transcripts, a feature that can prevent translation of the corresponding protein until an appropriate signal induces splicing and assembly of the mature mRNA¹⁸. Therefore, primer sets that flank intron four were designed and it was found that freshly isolated platelets (labeled, control and 0, in FIGS. 2B and 2C, respectively) predominantly contain TF pre-mRNA (FIGS. 2B and 2C, see also FIG. 4A and FIG. 8A), a finding that was consistent in >40 subjects.

It was next determined if platelets activated by adherence to fibrinogen and addition of thrombin splice TF pre-mRNA into a mature transcript. Splicing of TF pre-mRNA was detected at 5 min, neared completion by 1 h, and was sustained for at least 4 h after the platelets were activated (FIGS. 2B and 2C).

Other agonists such as ADP, collagen, or thrombin also induce TF pre-mRNA splicing in suspended platelets (FIG. 4). In particular, FIG. 4A shows TF and GAPDH mRNA expression in freshly isolated platelets (control), platelets that are activated in suspension by ADP (20 μM), or platelets adherent to fibrinogen in the presence of thrombin for the designated times. FIG. 4B shows TF mRNA expression in platelets that were left in suspension (control), platelets adherent to fibrinogen in the presence of thrombin, or platelets that are activated in suspension by ADP (20 μM), collagen (10 μg/ml), or thrombin (0.05 U/ml) for 30 min.

Anucleate platelets possess a functional spliceosome and can splice pre-mRNAs when activated establishing a mechanism for this sequence of events²⁰. To unequivocally demonstrate the cell source of pre- and mature TF mRNA species, the transcripts in individual megakaryocytes, proplatelets, and mature platelets were screened using indirect in situ PCR for intronic TF pre-mRNA (“noRT” indicates omission of the reverse transcriptase). TF pre-mRNA was present in the cytoplasm of hematopoietic stem cell-derived human megakaryocytes and proplatelets (FIG. 2D). Consistent with detection of intronic-rich message in platelet precursors, TF pre-mRNA was also found in freshly isolated platelets from circulating human blood (FIG. 2E, top left; quiescent platelets). It was also found that activated platelets (adhered to fibrinogen in the presence of thrombin for 1 hr) express TF mRNA (FIG. 2E, top right), confirming that the mature message is derived from platelets.

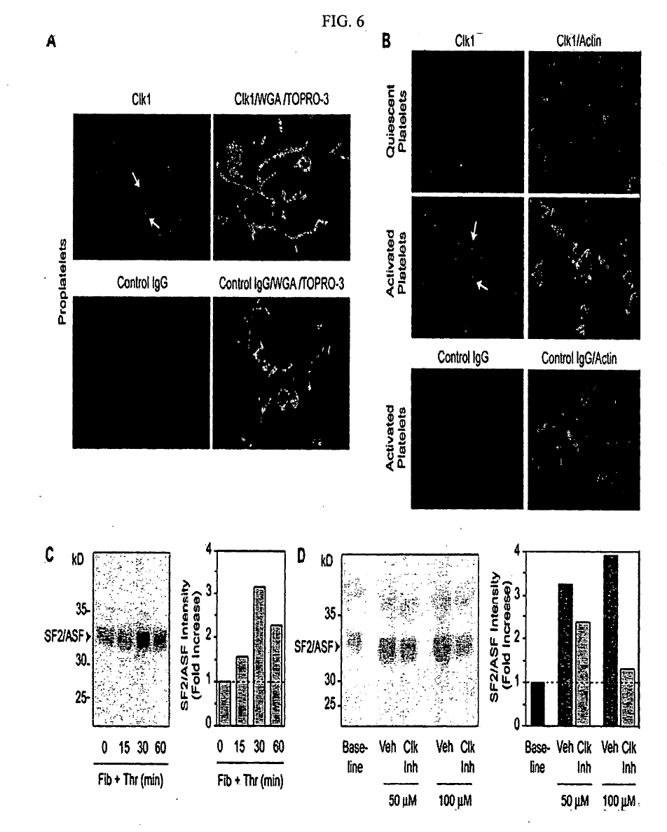

Since stimulated platelets use their splicing machinery to produce mature TF mRNA, it was next determined if TF-dependent procoagulant activity increased in activated cells. Freshly isolated platelets, at cell numbers (2×10⁹ total) that approximate those found in 5-10 ml of whole blood, did not possess significant levels (P<0.05) of procoagulant activity (FIGS. 5A and 5B). FIG. 5A is a time course of 0 to 60 minutes and FIG. 5B is a time course of 0 to 4 hours. For each of these figures, platelet cells were activated by adhesion to fibrinogen in the presence of thrombin. In FIG. 5B, procoagulant activity is shown for platelet membranes (Plts), platelet-derived microparticles (Mp), or platelet membranes and microparticles from the same sample (Plts+Mp). The data in FIGS. 5A and 5B is expressed as pM of TF per 2×10⁹ platelets and represent the mean±SEM of three independent experiments. As can be seen in these figures, procoagulant activity was markedly increased as early as 5 min after platelets adhered to fibrinogen in the presence of thrombin (FIG. 5A) and continued to accumulate in platelets and platelet-derived microparticles in a time-dependent fashion (FIG. 5B). Platelets activated in suspension with ADP, collagen, or thrombin also accumulated TF-dependent procoagulant activity. It was consistently observed that activated platelets possessed higher procoagulant activity than quiescent platelets in samples from different donors, but the magnitude was variable among subjects, ranging from a 2.8- to a 15.3-fold increase over baseline (mean increase over baseline 7.7±2.0). In contrast to activated platelets, monocytes (5×10⁶) stimulated with fibrinogen and thrombin did not generate appreciable procoagulant activity, although they did respond to lipopolysaccharide.

Next, the protein was analyzed by immunocytochemistry and TF was observed on the surfaces of activated platelets that had adhered and spread on immobilized fibrinogen in the presence of thrombin (2 hours), but not on quiescent platelets (FIG. 5C). Staining was detected on the surface of all the platelets, consistent with the detection of TF mRNA in every cell (FIG. 1E, top right).

Deletion of factor VIIa from the activity assay or incubation of platelets with a neutralizing anti-TF antibody significantly (P<0.05) reduced procoagulant activity in stimulated platelets (FIGS. 5D and 5E). The bars (n=4) in these panels show TF-dependent procoagulant activity in freshly isolated platelets or platelets activated as in FIG. 5B for 4 h in the presence or absence of factor VIIa (FIG. 2D) or a neutralizing antibody directed against TF (FIG. 5E). A single asterisk (*) indicates a statistically significant difference (P<0.05) in TF-dependent procoagulant activity between freshly isolated (baseline) and activated platelets; the double asterisk (**) represents a significant difference (P<0.05) between activated platelets under untreated or treated conditions.

This activity assay, however, evaluates factor Xa generation in the presence of supraphysiologic levels of exogenous factor VIIa⁷. Therefore, the affect of platelet-derived TF on plasma clotting times was determined. It was found that human plasma incubated with platelets activated with fibrinogen and thrombin for 5 min (left) or 2 h (right) formed clots at an accelerated rate compared with freshly isolated platelets, which by themselves had no appreciable effect on plasma clot formation (FIG. 5F). Clotting was significantly delayed in the presence of an inhibitory anti-TF antibody (FIG. 5F), indicating that TF generated by activated platelets is capable of accelerating in vitro clot formation in humans. Collectively, the studies depicted in FIG. 5 demonstrate that bioactive TF protein accumulates in platelets adherent to fibrinogen in the presence of thrombin. The white bars in FIGS. 5E and 5F represent activated platelets that were incubated with a neutralizing antibody directed against tissue factor (Anti-TF). The data represent the mean±SEM of six independent experiments.

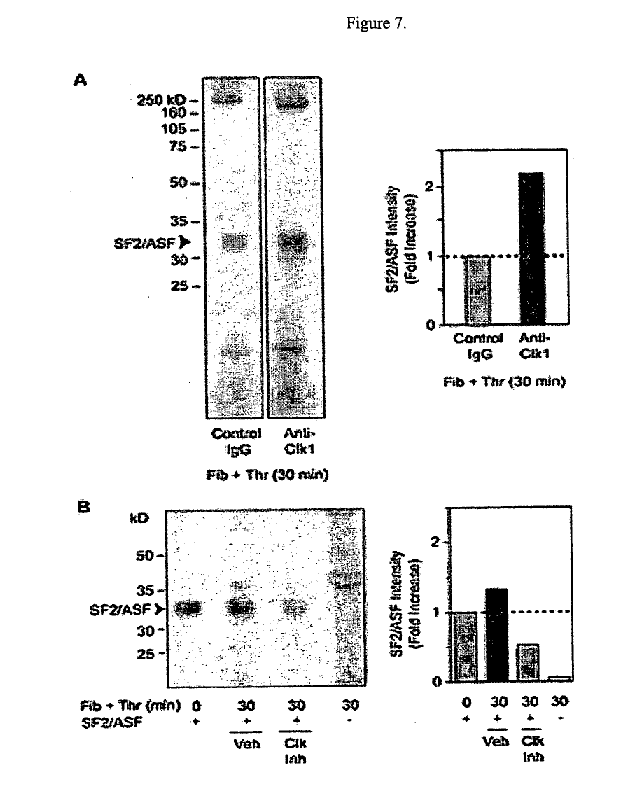

The intracellular signaling pathways that control TF pre-mRNA splicing and activity in platelets were not previously known¹⁸. In nucleated cells, serine-arginine (SR)-rich proteins regulate splicing, and it was recently shown that human platelets contain the SR family member SF2/ASF¹⁸. Thus, it was possible that platelets possess critical upstream kinases that regulate SF2/ASF activity and Clk family members were initially assayed because one of them, Clk1, contains an N-terminal region enriched in arginine-serine dipeptides (RS) that interacts with SF2/ASF²¹. Clk1 protein was found in the cytoplasm of mature megakaryocytes, in proplatelets that extend from the megakaryocytes (FIG. 6A, arrows), and in quiescent circulating platelets from human blood (FIG. 6B, arrows). In activated platelets, Clk1 was distributed to the tips of F-actin stress cables (FIG. 6B). Intracellular redistribution of Clk1 resembles the accumulation of vinculin in focal adhesion complexes of platelets that are spread and activated on immobilized fibrinogen²². Preliminary screens for other family members (Clk2, 3, and 4) were negative, suggesting that Clk1 is the primary Clk in mature, circulating platelets.

In FIGS. 6A and 6B, megakaryocytes with proplatelet extensions were stained with anti-Clk1 (top left) or control IgG (bottom left). The right panels are overlays where anti-Clk1 or IgG (in red), wheat germ agglutinin (in green), and topro-3 (staining nuclei in blue) are all shown. In FIG. 6B platelets were left quiescent (top) or activated by adherence to immobilized fibrinogen in the presence of thrombin for 1 h (middle and bottom). The cells were stained with anti-Clk1 (top and middle) or control IgG (bottom). The right panels are overlays in showing anti-Clk1 or IgG, polymerized actin, and colocalization of the two markers.

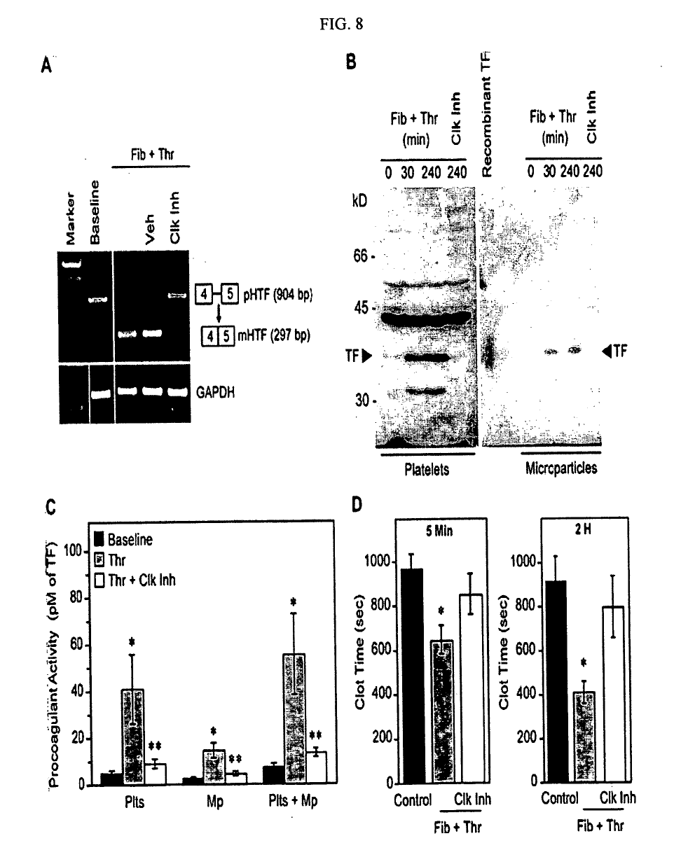

Clk1 is a dual-specificity kinase that autophosphorylates on tyrosine, serine, and threonine and contains a SR (serine-arginine) domain that phosphorylates SR proteins⁸⁴. In nucleated cells, Clk1 directly phosphorylates SF2/ASF, promotes spliceosome assembly and thereby facilitates pre-mRNA splicing, and alters the intracellular localization patterns of SR proteins. Therefore, endogenous platelet Clk1 was captured by immunoprecipitation and assayed for regulation of SF2/ASF phosphorylation. FIG. 6C shows a time course of Clk1-dependent SF2/ASF phosphorylation in activated platelet lysates. Clk1 from activated platelets (grown on immobilized fibrinogen in the presence of thrombin for 30 minutes) markedly increased SF2/ASF phosphorylation (FIG. 6C and FIG. 7A). Increased SF2/ASF phosphorylation was not seen when control IgG was used as the immunoprecipitating reagent (FIG. 7A).

Platelets (FIGS. 6C and 6D) were activated as in FIG. 6B, lysed, and immune complex kinase assays for Clk1 activity were performed with recombinant SF2/ASF as the substrate. The bars for FIGS. 6C, 6D, 7A and 7B represent fold increases in SF2/ASF phosphorylation over baseline as estimated by densitometry.

A benzothiazole compound, Tg003, suppresses Clk1-catalyzed phosphorylation and thereby inhibits SF2/ASF-dependent splicing of in vitro-transcribed pre-mRNAs in immortalized cell lines²³. FIG. 6D shows Clk1-dependent SF2/ASF phosphorylation in freshly isolated platelet lysates or platelets that were pretreated with vehicle or Tg003 (Clk Inh) (lanes 2-5). In stimulated platelets, Tg003, but not its vehicle, suppressed Clk1-dependent SF2/ASF phosphorylation (FIGS. 6D and 7B), consistent with previous characterization of the inhibitor in other cells²³.

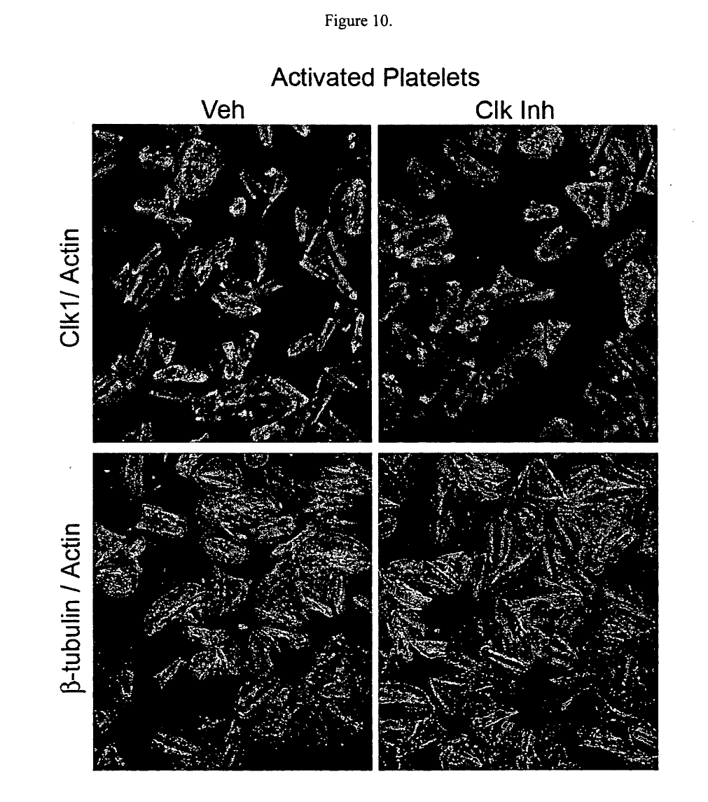

It was next determined if interruption of signaling from Clk1 to SF2/ASF modulates activation-dependent splicing and found that Tg003 prevented processing of TF pre-mRNA in activated platelets (FIG. 8A). In FIG. 5A freshly isolate platelets or platelets adhered to immobilized fibrinogen and coactivated with thrombin for 2 hours were used and treated with vehicle (Veh) or Tg003 (Clk Inh). FIG. 5B is a western blot showing TF protein expression in platelets and platelet-derived microparticles, where the platelets were left quiescent (baseline) or adhered to immobilized fibrinogen and acoactivated with thrombin for 30 minutes or 4 hours in the presence or absence of Tg003. Recombinant TF served as a positive control. This demonstrates that a Clk1-dependent splicing inhibitor also blocks the expression of TF protein in activated platelets and platelet-derived microparticles (FIG. 8B). Consistent with its effect on protein, platelets activated as in FIG. 5B were used to show that Tg003 significantly (P<0.05) reduced TF-dependent procoagulant activity in stimulated platelets (FIG. 5C). Likewise, membranes isolated from freshly isolated platelets (control) or platelets activated for 5 min. or 2 hours and pretreated with Tg003 or vehicle (DMSO) demonstrated that Tg003 delayed the onset of plasma clot formation (FIG. 4D). Puromycin, an inhibitor of mRNA translation, also significantly (P<0.05) reduced TF-dependent procoagulant activity demonstrating the increases were caused by de novo protein synthesis (FIG. 9).

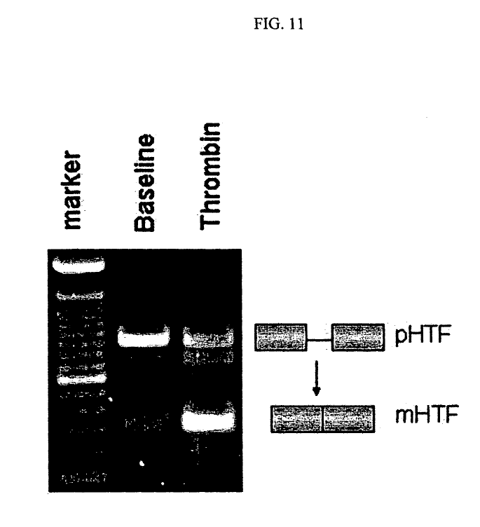

Although Tg003 blocked SF2/ASE phosphorylation (FIG. 6C) and the expression of TF protein in activated platelets (FIG. 8), it had no effect on other platelet functional responses that included cellular adherence and spreading, actin polymerization, organization of O-tubulin, or the redistribution of Clk1 to focal adhesion complexes (FIG. 10). These data suggest that the Clk1 signaling pathway primarily interfaces with the splicing machinery in platelets.

### Materials and Methods

CD34+ stem cells were isolated from human umbilical cord blood and were differentiated into megakaryocytes that produce proplatelets using methods that we have previously described¹⁸. Leukocyte-depleted human platelets were isolated from healthy volunteers using previously described methods^{18,29}. For all of the platelet studies, residual leukocytes were removed from the washed preparations by CD45+ bead selection as previously described¹. Flow cytometric gating of the entire cell population demonstrated that CD45-depleted platelet preparations did not contain CD45, P-selectin glycoprotein-1 (PSGL-1), or CD14-positive cells (FIG. S1). Platelets were resuspended in M199 serum-free culture medium^{1,2}. Platelets were left quiescent or allowed to adhere to immobilized human fibrinogen (Calbiochem) in the presence of thrombin (0.05 U/ml; Sigma-Aldrich). Select studies were also conducted with suspended platelets that were activated with ADP, collagen, or thrombin. For splicing inhibition, platelets were preincubated for 30 min with Tg003 (Calbiochem) or DMSO. For translational inhibition, platelets were isolated and preincubated for 2 h in the presence of vehicle (water) or puromycin as previously described².

For most of the TF pre-mRNA studies, primers that targeted sequences in exon four (5′-CTCGGACAGCCAACAATTCAG-3′; SEQ ID NO: 1) and five (5′-CGGGCTGTCTGTACTCTTCC-3′; SEQ ID NO: 2), and thus spanned intron four, were used to determine endogenous splicing of TF pre-mRNA in platelets. Indirect in situ hybridization or direct in situ PCR was used to detect TF pre-mRNA in megakaryocytes and platelets as previously described¹⁸. Primers that detected alternatively spliced human TF (asHTF) were used as previously described³. To detect full-length mature mRNA for human TF (mHTF) in activated platelets, primers were designed that targeted sequences in exon one (5′-CCAACTGGAGACATGGAGAC-3′ SEQ ID NO: 3) and exon six (5′-CAGTAGCTCCAACAGTGCTTCC-3′; SEQ ID NO: 4). Indirect in situ hybridization or direct in situ PCR was used to detect TF pre-mRNA in megakaryocytes and platelets, respectively¹. Primers specific for intron four (5′-ACCCATTTCTTCCCCAATTC-3′ (SEQ ID NO: 5) and 5′-GTGCCTGGGATCCTCAATAG-3′ (SEQ ID NO: 6)) were used to generate DIG-labeled intronic probes for the indirect in situ PCR and direct in situ PCR experiments. For platelets that were adherent to fibrinogen in the presence of thrombin, the generated cDNA was amplified in the presence of DIG-labeled dNTP using primers that targeted exons three (5′-CTCCCCAGAGTTCACACCTTAC-3′; SEQ ID NO: 7) and five (5′-CGGGCTGTCTGTACTCTTCC-3′; SEQ ID NO: 8), respectively. These exonic primers allowed detection of the spliced product (331 bp), but not the unspliced product (3,635 bp), by normal PCR methods.

Detailed strategies for protein detection by flow cytometry, Western blot analysis, and immunocytochemistry have been previously published^{18,29}. A polyclonal antibody against human TF (pAb CL 20150A; Affinity Biologicals) was used under nonreducing conditions to detect TF protein by Western analysis. Antibodies directed against TF (mAb 550252; BD Biosciences), Clk family members (Abgent), or β-tubulin (Sigma-Aldrich) were used for immunocytochemical studies. For the in situ experiments, polymerized actin was detected with Alexa fluor 488 phalloidin (Molecular Probes). Specificity was confirmed by parallel studies with nonimmune IgG or deletion of the primary antibody. Detailed strategies for protein detection by Western analysis and immunocytochemistry have been previously published^{1,2}. Flow cytometric analysis of surface-expressed CD14, CD45, and CD162 (PSGL-1) was performed on washed platelet preparations before and after CD45 depletion, on CD45 positively selected cells, and on purified monocytes (FIG. 1).

TF-dependent procoagulant activity was calculated with an Actichrome TF assay (American Diagnostica) as previously described¹³. A total of 2×10⁹ freshly isolated CD45-depleted platelets, a value that approximates the number of platelets found in 10 ml of whole blood obtained from healthy subjects, was resuspended in M199 media. Platelets were left quiescent or activated in the presence or absence of Tg003. At the end of each experimental point, the platelets were immediately centrifuged at 15,500 g for 4 min at 4° C. The supernatants were collected and recentrifiged at 100,000 g for 90 min at 4° C. to pellet microparticles. In parallel, the platelet pellets were resuspended in ice-cold 250 mM sucrose that was suspended in 10 mM of PBS that contained a broad band protease inhibitor cocktail. After a brief sonication to disrupt the cells, the platelets were centrifuged for 15 min at 420 g (4° C.) to separate the sedimented cellular components from the supernatant-rich membranes. The supernatants were recentrifuged at 20,800 g for 30 min (4° C.) to pellet the membrane proteins. Intact cellular membranes and microparticles were immediately placed in 25 μl of kit assay buffer and TF-dependent procoagulant activity was calculated. In separate studies, we found that disruption of platelet membranes or microparticles by standard detergent lysis markedly reduced activity. To demonstrate the specificity of the assay for TF procoagulant activity, some samples were preincubated with a neutralizing TF antibody (pAb 4502; American Diagnostica). Factor VIIa was also eliminated from the reaction. The data are displayed as pM of TF per 2×10⁹ platelets.

Immune complex kinase assay for Clk1 activity. Clk1 activity in platelets was determined using an immune complex kinase assay. An antibody against Clk1 was used for immunoprecipitation of the protein. Nonimmune rabbit IgG was used as a control, and in select experiments recombinant SF2/ASF was removed from the assays to screen for nonspecific incorporation of radiolabeled phosphate (FIGS. 7A and B). Kinase assays were performed by addition of recombinant SF2/ASF (Protein One) in the presence of γ-[³²P]ATP (MP Biomedicals). At the end of this incubation period, the agarose beads and immune complexes were removed by centrifugation, and the unbound sample, which contained SF2/ASF, was resolved by SDS-PAGE.

Statistical analyses. ANOVA was conducted to identify differences that existed among multiple experimental groups. If significant differences were found, a Student-Newman-Keuls post-hoc procedure was used to determine the location of the difference. For all of the analyses, P<0.05 was considered statistically significant.

Tg003 ((Z)-1-(3-Ethyl-5-methoxy-2,3-dihydrobenzothiazol-2-ylidene) propan-2-one) is an example of a Clk1 inhibitor that is commercially available. It is a cell-permeable dihydrobenzothiazolo compound reported to be a potent, specific, reversible, and ATP-competitive inhibitor of Clk-family of kinases (Ki=10 nM for mClk1/Sty; IC50=15 nM, 20 nM, 200 nM, and >10 μM for mClk4, mClk1, mClk2, and mClk3, respectively). It does not affect the activities of SRPK1, SRPK2, PKA, or PKC at concentrations up to 1 μM. See U.S. Patent Pub. 20050171026, which is incorporated by reference.

### EXAMPLE I

It has been found that aspirin and heparin do not block signal-dependent TF pre-mRNA splicing in the elderly (FIG. 11) or IL-1β pre-mRNA splicing in young subjects.

### EXAMPLE II

A number of commonly used drugs in the elderly have not been found to modulate TF pre-mRNA splicing and/or TF-dependent coagulation activity in platelet cells. Therefore, additional medications that are currently available may be screened for the ability to affect post-transcriptional gene expression in platelets, for example, the ability to modulate TF pre-mRNA splicing and/or TF-dependent coagulation activity. In particular, those medications that are prescribed for prevention of thrombotic and vascular events may be screened.

Thus, the in vivo effects of three common medications, aspirin, clopidogrel bisulfate, and HMG-CoA reductase inhibitors (or “statins”) on pre-mRNA splicing may be determined. Optionally, in vitro analyses may be done, for example, with aspirin and ADP receptor antagonists. It will be recognized that aspirin, clopidogrel bisulfate, and statins are merely a subset of the prescribed medications, and that any compound or drug may be tested or screened according to the invention.

For studies directed to clopidogrel bisulfate, and statin, or larger studies directed to aspirin, patients are recruited and blood samples obtained. IL-1β and TF pre-mRNA splicing and corresponding protein synthesis are then measured in appropriate cell populations. The results generated from the medicated elderly donors are compared to those from the non-medicated, healthy elderly subjects.

Clinical trials for primary and secondary prevention of arterial thrombotic events have demonstrated that aspirin, which affects platelet aggregation by irreversibly inhibiting cyclooxygenase-1 (COX-1) and subsequently blocking thromboxane A2 (TxA2) production, is effective at both low and high doses¹⁵⁶. Patients who are taking aspirin alone for at least three months are candidates for the study. Doses between 81 mg and 325 mg daily are accepted because clinical evidence suggests the anti-thrombotic effects of aspirin appear to be similar in this dosing range¹⁵⁷. Medication compliance is assessed by activating platelets and measuring TxB2 generation in the supernatants²⁴. In addition, pill counts will be performed and patients who are taking >80% of their prescribed daily dose (a “medication possession ratio”, MPR, >0.80) are considered compliant. These criteria are commonly used in large clinical trials and are an accepted method of confirming medication compliance^{158,159}.

In addition, the in vitro effect of aspirin on pre-mRNA splicing may be measured. Platelets may be pretreated ex vivo with aspirin and activation-dependent generation of TxB2 is examined in parallel with TF pre-mRNA splicing to confirm the efficacy of in vitro aspirin treatment²⁴.

Clopidogrel bisulfate (“clopidogrel”) selectively and irreversibly inhibits the binding of ADP to its platelet receptor, subsequently inhibiting platelet aggregation¹⁶⁰. Inhibition of platelet aggregation occurs as early as 2 hours after a single dose of clopidogrel and a steady state is reached between days 3 and 7. Secondary prevention trials have shown that clopidogrel reduces the incidence of cardiovascular events¹⁶¹.

Because many patients on clopidogrel also take aspirin and the initial data (FIG. 20) indicates that aspirin does not block pre-mRNA splicing events, the effects of clopidogrel and aspirin therapy on post-transcriptional gene expression in platelets may be examined. Patients who are taking aspirin (at doses between 81 mg and 325 mg daily) and clopidogrel 75 mg daily (a standard dose) for a minimum of three months are enrolled. Medication compliance is determined using standard strategies. Optionally, ADP-induced platelet aggregation may be examined.

Clopidogrel cannot be studied in vitro because it is a pro-drug that requires activation via the liver¹⁶⁰. Nevertheless, other ADP-receptor antagonists may be studied in vitro. Therefore, studies may be conducted to assess the effects of ADP-receptor antagonists, or aspirin in combination with an ADP-receptor antagonist, on pre-mRNA splicing events in vitro. Platelets from non-medicated donors are pretreated ex vivo with these compounds using concentrations that approximate in vivo dosing. In each study, ADP-dependent aggregation and activation-dependent generation of TxB2 is examined in parallel with TF pre-mRNA splicing to confirm the efficacy of in vitro ADP-receptor antagonist and aspirin treatment, respectively. Identification of an ADP-receptor antagonist capable of modulating TF pre-mRNA splicing in platelets indicates that such an ADP-receptor antagonist could be useful in the treatment of other coagulation diseases and/or that it may be more effective in combination with other drugs.

Statins are inhibitors of 3-hydroxy-3-methylglutaryl-coenzyme A (HMG-CoA) reductase and are used clinically to reduce levels of low-density-lipoproteins (LDL)¹⁶². Both primary and secondary prevention trials have demonstrated that when used at “Standard doses”, statins reduce LDL levels by 25-38% and are associated with a reduction in cardiovascular events of 27-37%^{163,164}. In addition, statins are thought to stabilize plaques, reduce platelet hyperreactivity and inhibit TF expression^{165,166}. Furthermore, clinical trials demonstrate an added benefit of statins plus aspirin compared to statins alone¹⁶⁷. To determine the effect of statins on TF pre-mRNA splicing or platelet TF activation, elderly patients who are taking aspirin (at doses between 81 mg and 325 mg daily) and any statin prescribed at a “standard dose” or higher (fluvastatin 40-80 mg daily, lovastatin 40 mg daily or higher, pravastatin 40 mg daily, simvastatin 20 mg daily or higher, atorvastatin 10 mg daily or higher, and rosuvastatin 5 mg daily or higher), for 3 months or longer, are enrolled. Compliance is assessed and may include reviewing recent lipid panels. In vitro analyses using statins may not be possible since they are pro-drugs. Therefore, in vivo analysis may be preferable.

Based on the initial data, it is anticipate that aspirin will have no effect on pre-mRNA splicing and associated events. Similar results in patients treated with aspirin in combination with clopidogrel or statins are anticipated. However, if clopidogrel or one or more statins is found to modulate pre-mRNA splicing events in the presence of aspirin, a subset of patients that are solely using that drug are recruited to confirm the results.

For the quantitative assays, the mean±SEM and ANOVA are used to identify differences among the various experimental groups. The location of each difference is identified by post-hoc tests^{13,22,25,143}. Statistical significance is typically set at p<0.05. In addition, approximately 5-6 experiments are typically performed for the non-quantitative assays (e.g., western blots, immunocytochemistry, etc.) so that representative conclusions may be made with confidence.

ANOVA analysis may also be used to perform a subgroup analysis based on age (i.e., 65-79 and >80 yr) to screen for trends and identify significant differences between age groups.

### EXAMPLE III

Therapeutic target recognition, development and validation is performed on a library of compounds. Freshly isolated CD45-depleted platelets are resuspended in M199 media. Platelets are left quiescent or activated (e.g., incubated with thrombin) in the presence or absence of a test compound, such as those described in U.S. Pat. Nos. 5,292,765 and/or 4,051,141. At the end of each experimental point, the platelets are centrifuged at 15,500 g for 4 min at 4° C. Platelet pellets are resuspended in cold 250 mM sucrose that is suspended in 10 mM of PBS containing a broad band protease inhibitor cocktail. After a brief sonication to disrupt the cells, the platelets are centrifuged for 15 min at 420 g (4° C.) to separate the sedimented cellular components from the supernatant-rich membranes. The supernatants are recentrifuged at 20,800 g for 30 min (4° C.) to pellet the membrane proteins. Intact cellular membranes and microparticles are placed in 25 μl of Actichrome TF kit assay buffer and TF-dependent procoagulant activity is measured. Tg003 may be used as a positive control for inhibition.

One or more test compounds are identified as inhibiting TF-dependent coagulation in platelet cells. Such test compounds are then tested for inhibition of TF pre-mRNA splicing and/or Clk1 activation. One or more test compounds identified as inhibiting TF pre-mRNA splicing are then further validated for efficacy as a therapeutic agent in the treatment of a coagulation disorder.

### EXAMPLE IV

96-, 384- or 1536-well white opaque plates are used to screen potential Clk1 modulators. A phosphoserine specific antibody is conjugated to AlphaScreen Protein A Acceptor beads, available from PerkinElmer. Streptavidin-coated AlphaScreen Donor beads, available from PerkinElmer, Biotinylated RS-peptide (RSRSRSRSRSR; SEQ ID NO XX), Activated or quiescent amino terminal GST tagged Clk1 Kinase, available from Cell Signalling Technology®, and a compound to be tested are added to kinase buffer (60 mM HEPES-NaOH, pH 7.5, 3 mM MgCl₂, 3 mM MnCl₂, 3 μM Na-orthovanadate, 1.2 mM DTT, 50 ng/μl PEG_{20,000}, 40 ng/μl, and 10 mM ATP). The order of addition is adjusted to facilitate high-throughput screening of the test compounds. The reaction is incubated for an appropriate period of time and the reaction is stopped. Modulation of Clk1 activity may be measured using an appropriate plate reader (see, A Practical Guide to working with AlphaScreen, PerkinElmer, Inc. 2004).

Other chemiluminescent or fluorescent detection systems may be used, including, but not limited to, LANCE® (PerkinElmer), TRuLight® (Calbiochem), and Kinase-Glo (Promega).

### EXAMPLE V

Identification of agents that affect splicing-meditated platelet activation may also be performed by direct analysis of RNA splicing in platelet extracts. In vitro splicing assays may be carried out using extracts of activated platelets and a reporter construct, such as a TF reporter transcript, as interleukin 1β, as a substrate. RNA substrates may be synthesized by in vitro transcription according to established protocols, for example, using T3 polymerase and commercially available reagents, such as the MEGAscript® system (Ambion). Reaction mixtures typically contain platelet extract, RNA substrate, and a compound to be tested. Specific detection of spliced RNA product may be accomplished using quantitative reverse transcriptase PCR (RT-PCR) with fluorescent detection. Reagents for quantitative RT-PCR, with detection using either FRET probe-based or SYBR Green based systems, are commercially available from a number of sources, including Sigma and Roche Molecular Systems, and are compatible with assay automation.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein, including:
1. Jurk, K., and B. E. Kehrel. Platelets: physiology and biochemistry. Semin. Thromb. Hemost. 2005; 31:381-392.
2. Ruggeri, Z. M. Platelets in atherothrombosis. Nat. Med. 2002; 8:1227-1234.
3. Roberts, H. R., M. Hoff man, and D. M. Monroe. A cell-based model of thrombin generation. Semin. Thromb. Hemost. 2006; 32(Suppl 1):32-38.
4. Lentz, B. R. Exposure of platelet membrane phosphatidylserine regulates blood coagulation. Prog. Lipid Res. 2003; 42:423-438.
5. Morrissey, J. H., et al. Factor VIIa-tissue factor: functional importance of protein-membrane interactions. Thromb. Haemost. 1997; 78:112-116.
6. Mackman, N. Role of tissue factor in hemostasis, thrombosis, and vascular development. Arterioscler. Thromb. Vase. Biol. 2004; 24:1015-1022.
7. Butenas, S., et al Tissue factor activity in whole blood. Blood. 2005; 105:2764-2770.
8. Camera, M., et al Platelet activation induces cell-surface immunoreactive tissue factor expression, which is modulated differently by antiplatelet drugs. Arterioscler. Thromb. Vasc. Biol. 2003; 23:1690-1696.
9. Muller, I., et al. Intravascular tissue factor initiates coagulation via circulating microvesicles and platelets. FASEB J. 2003; 17:476-478.
10. Osterud, B., and E. Bjorklid. Sources of tissue factor. Semin. Thromb. Hemost. 2006; 32:11-23.
11. Siddiqui, F. A., et al. The presence and release of tissue factor from human platelets, Platelets. 2002; 13:247-253.
12. Zillmann, A., et al. Platelet-associated tissue factor contributes to the collagen-triggered activation of blood coagulation. Biochem. Biophys. Res. Commun. 2001; 281:603-609.
13. Del Conde, I., C. N. Shrimpton, P. Thiagarajan, and J. A. Lopez. Tissue-factor-bearing microvesicles arise from lipid rafts and fuse with activated platelets to initiate coagulation. Blood. 2005; 106:11604-1611.
14. Falati, S., et al. Accumulation of tissue factor into developing thrombi in vivo is dependent upon micro particle P-selectin glycoprotein ligand I and platelet P-selectin. J. Exp. Med. 2003; 197:1585-1598.
15. Rauch, U., et al. Transfer of tissue factor from leukocytes to platelets is mediated by CD15 and tissue factor. Blood. 2000; 96:170-175.
16. Pereira, J., et al. Circulating platelet-derived microparticles in systemic lupus erythematosus. Association with increased thrombin generation and procoagulant state. Thromb. Haemost. 2006; 95:94-99.
17. Reininger, A. J., e al. Mechanism of platelet adhesion to von Willebrand factor and microparticle formation under high shear stress. Blood. 2006; 107:3537-3545.
18. Denis, M. M., et al. Escaping the nuclear confines: signal-dependent pre-mRNA splicing in anucleate platelets. Cell. 2005; 122:379-391.
19. Bogdanov, V. Y., et al. Alternatively spliced human tissue factor: a circulating, soluble, thrombogenic protein. Nat. Med. 2003; 9:458-462.
20. Meshorer, E., and T. Misteli. Splicing misplaced. Cell. 2005; 122:317-318,
21. Colwill, K., et al. The Clk/Sty protein kinase phosphorylates SR splicing factors and regulates their intranuclear distribution. EMBO J. 1996; 15:265-275.
22. Leng, L., H. Kashiwagi, X. D. Ken, and S. J. Shattil. RhoA and the function of platelet integrin alphaIIbbeta3, Blood. 1998; 91:4206-4215.
23. Muraki, M., et 1. Manipulation of alternative splicing by a newly developed inhibitor of Clks. J. Biol. Chem. 2004; 279:24246-24254.
24. Weyrich, A. S., et al. Change in protein phenotype without a nucleus: translational control in platelets. Semin. Thromb. Hemost. 2004; 30:491-498.
25. Chou, J., et al. Hematopoietic cell-derived microparticle tissue factor contributes to fibrin formation during thrombus propagation. Blood. 2004; 104:3190-3197.
26. Ni, H., et al. Persistence of platelet thrombus formation in arterioles of mice lacking both von Willebrand factor and fibrinogen. J. Clin. Invest. 2000; 106:385-392.
27. Fernandez, P. M., S. R. Patierno, and F. R. Rickles. Tissue factor and fibrin in tumor angiogenesis. Semin. Thromb. Hemost. 2004; 30:31-44.
28. Levi, M., T. van der Poll, and H. ten Cate. Tissue factor in infection and severe inflammation. Semin. Thromb. Hemost. 2006; 32:33-39.
29. Weyrich, A. S., et al. Signal-dependent translation of a regulatory protein, Bcl-3, in activated human platelets. Proc. Natl. Acad. Sci. USA. 1998; 95:5556-5561.
30. Drake T A, Cheng J, Chang A, Taylor F B, Jr. Expression of tissue factor, thrombomodulin, and E-selectin in baboons with lethal Escherichia coli sepsis. Am J Pathol. 1993; 142:1458-1470.
31. Italiano J E, Jr., Shivdasani R A. Megakaryocytes and beyond: the birth of platelets. J Thromb Haemost. 2003; 1:1174-1182.

What is claimed is:

1. A method for identifying a compound that modulates pre-mRNA splicing in a platelet cell, the method comprising:
contacting a platelet cell with a compound;
preparing an RNA sample from the platelet cell;
amplifying the RNA Sample; and
assaying mRNA for excision of an intron.

2. The method according to claim 1, wherein amplifying the RNA sample comprises conducting a polymerase chain reaction.

3. The method according to claim 1, wherein assaying mRNA for excision of an intron comprises assaying tissue factor mRNA.

4. The method according to claim 3, further comprising assaying for excision of intron four from a tissue factor pre-mRNA.

5. The method according to claim 4, comprising amplifying the RNA sample using SEQ ID NO: 1 and SEQ ID NO: 2.

6. The method according to claim 3, comprising amplifying the RNA using a first primer specific to exon 4 and a second primer specific to exon 5.

7. The method according to claim 1, wherein assaying mRNA for excision of an intron comprises assaying by gel electrophoresis.

8. The method according to claim 1, further comprising quantifying a degree of tissue factor pre-mRNA splicing.

9. The method according to claim 1, wherein contacting a platelet cell with a compound comprises screening a library of compounds.

10. The method according to claim 9, further comprising selecting at least one compound from the library of compounds.

11. The method according to claim 9, identifying one or more compounds that inhibit tissue factor pre-mRNA splicing and validating the one or more compounds identified as inhibiting tissue factor pre-mRNA splicing for efficacy as a therapeutic agent in the treatment of a coagulation disorder.

12. A method for identifying a compound that modulates Clk1 activity, the method comprising:
contacting a Clk1 preparation containing a reporter capable of being phosphorylated by Clk1 and labeled phosphate with a compound;
incubating the Clk1 preparation and the compound; and
determining the effect of the compound on the ability of Clk1 to phosphorylate the reporter.

13. The method according to claim 12, wherein determining the effect of the compound on the ability of Clk1 to phosphorylate the reporter comprises assaying phosphorylation of a SF2/ASF splicing factor.

14. The method according to claim 12, comprising screening a library of compounds.

15. A method for identifying a compound that modulates TF-dependent procoagulant activity in a platelet cell, the method comprising:
contacting a platelet cell with a compound;
isolating a platelet membrane from the platelet cell;
adding the platelet membrane to human plasma;
initiating clotting;
measuring clotting time; and
determining the affect of the compound on clotting time.

16. The method according to claim 15, further comprising adding a tissue factor pre-mRNA splicing stimulant.

17. The method according to claim 16, further comprising screening a library of compounds for an activity that inhibits an increase in clotting due to the stimulant.

18. The method according to claim 17, further comprising selecting at least one compound from the library of compounds.

19. A method of treating or preventing a disease or disorder associated with coagulation in a subject comprising administering to a subject an effective amount of a compound that inhibits CLK-1 activity.

20. The method according to claim 19, wherein the compound is (Z)-1-(3-Ethyl-5-methoxy-2,3-dihydrobenzothiazol-2-ylidene)propan-2-one.

21. The method according to claim 19, wherein the disease or disorder is sepsis or venous thromboembosis.

22. A method of inhibiting production of tissue factor in a platelet cell of a subject comprising administering to a subject an effective amount of a compound that inhibits CLK-1 activity in a platelet cell of the subject.


SEQUENCE LISTING (8 sequences)

SEQ ID NO: 1 - DNA, 21 bp, Artificial
  TF pre-mRNA exon four primer
ctcggacagccaacaattcag

SEQ ID NO: 2 - DNA, 20 bp, Artificial
  TF pre-mRNA exon five primer
cgggctgtctgtactcttcc

SEQ ID NO: 3 - DNA, 21 bp, Artificial
  TF pre-mRNA exon 1 primer
ccaactggtagacatggagac

SEQ ID NO: 4 - DNA, 22 bp, Artificial
  TF pre-mRNA exon six primer
cagtagctccaacagtgcttcc

SEQ ID NO: 5 - DNA, 20 bp, Artificial
  in situ primer for intron four
acccatttcttccccaattc

SEQ ID NO: 6 - DNA, 20 bp, Artificial
  in situ primer for intron four
gtgcctgggatcctcaatag

SEQ ID NO: 7 - DNA, 22 bp, Artificial
  In situ exon 3 primer
ctccccagagttcacaccttac

SEQ ID NO: 8 - DNA, 20 bp, Artificial
  second exon five primer
cgggctgtctgtactcttcc